(19) United States
(12) Patent Application Publication
(10) Pub. No.: US 2021/0069354 A1
(43) Pub. Date: Mar. 11, 2021

(54) METHOD OF TRANSPORTING AN AGENT ACROSS BLOOD-BRAIN, BLOOD-COCHLEAR OR BLOOD-CEREBROSPINAL FLUID BARRIER

(71) Applicants: Oklahoma Medical Research Foundation, Oklahoma City, OK (US); Hough Ear Institute, Oklahoma City, OK (US)

(72) Inventors: Richard D. Kopke, Oklahoma City, OK (US); Rheal A. Towner, Piedmont, OK (US)

(73) Assignees: Oklahoma Medical Research Foundation; Hough Ear Institute

(21) Appl. No.: 16/951,736

(22) Filed: Nov. 18, 2020

Related U.S. Application Data
(63) Continuation of application No. 15/760,779, filed on Mar. 16, 2018.
(60) Provisional application No. 62/220,575, filed on Sep. 18, 2015.

(51) Int. Cl. A61K 49/10; A61K 31/10; A61K 31/15; A61K 47/20

(52) U.S. Cl. CPC A61K 49/10; A61K 47/20; A61K 31/15; A61K 31/10

(57) ABSTRACT

Disclosed here is a method of transporting a therapeutic or diagnostic agent across a blood-brain barrier or a blood-cochlear barrier or a blood-cerebrospinal fluid barrier of a subject, comprising administering to a subject an amount of a therapeutic and/or diagnostic agent, along with an amount of 2,4-disulfonyl a-phenyl tertiary butyl nitrone (2,4-DSPBN), said therapeutic and/or diagnostic agent being characterized as being unable or poorly able, in the absence of said amount of 2,4-DSPBN, to cross the blood-brain barrier or the blood-cochlear barrier or the blood-cerebrospinal fluid barrier of said subject.

### CROSS-REFERENCE TO RELATED APPLICATIONS

This application is a continuation of U.S. application Ser. No. 15/760,779 filed Mar. 16, 2018, which is a 371 of International Application No. PCT/US2016/051632 filed Sep. 14, 2016, which claims the benefit of U.S. Provisional Patent Application No. 62/220,575 filed Sep. 18, 2015, the contents of which are incorporated herein by reference in its entirety.

### BACKGROUND

The blood-brain barrier (BBB) is a highly regulated barrier designed to regulate brain homeostasis while permitting selective transport of molecules that are essential for brain function. The BBB, however, prevents entry into the brain of most pharmaceuticals from the blood. The presence of the BBB makes it difficult to develop new treatments of brain diseases, or new radiopharmaceuticals for neuroimaging of brain.

Access to the inner ear region is regulated by the blood-cochlear barrier (BCB), which is anatomically and functionally similar to the BBB. The presence of the BCB prevents many pharmaceuticals from gaining access to the inner ear region, thus hampering their potentially therapeutic effects for treating inner ear injuries.

The blood-cerebrospinal fluid barrier (BCSFB) is a barrier located at the tight junctions that surround and connect the cuboidal epithelial cells on the surface of the choroid plexus. The BCSFB regulates access to the cerebrospinal-fluid and is distinct from the BBB.

Therefore, a need exists for innovative methods of transporting a therapeutic or diagnostic agent across the BBB or the BCB or the BCSFB, thereby improving its therapeutic or diagnostic effects.

### SUMMARY

One aspect of the invention described herein relates to a method of transporting a therapeutic and/or diagnostic agent across a blood-brain barrier or a blood-cochlear barrier or a blood-cerebrospinal fluid barrier of a subject in need thereof, comprising administering to a subject an amount of a therapeutic and/or diagnostic agent, along with an amount of 2,4-disulfonyl α-phenyl tertiary butyl nitrone (2,4-DSPBN), said therapeutic and/or diagnostic agent being characterized as being unable or poorly able, in the absence of said amount of 2,4-DSPBN, to cross the blood-brain barrier or the blood-cochlear barrier or the blood-cerebrospinal fluid barrier of said subject.

In some embodiments, the 2,4-DSPBN and the diagnostic and/or therapeutic agent are co-administered as a mixture. In some embodiments, the 2,4-DSPBN and the diagnostic and/or therapeutic agent are co-administered as a covalently- or noncovalently-bound conjugate. In some embodiments, the 2,4-DSPBN and the diagnostic and/or therapeutic agent are administered sequentially as distinct dosage forms.

In some embodiments, the 2,4-DSPBN and the diagnostic and/or therapeutic agent are administered orally into the subject. In some embodiments, the 2,4-DSPBN and the diagnostic and/or therapeutic agent are administered intravenously, subcutaneously, by inhalation, sublingually, subdermally, intrathecally, or locally within the ear.

In some embodiments, the administration of the 2,4-DSPBN increases permeability of the blood-brain barrier of said subject. In some embodiments, the administration of the 2,4-DSPBN increases permeability of the blood-cochlear barrier of said subject. In some embodiments, the administration of the 2,4-DSPBN increases permeability of the blood-cerebrospinal fluid barrier of said subject.

In some embodiments, the subject is a human patient suffering from an otologic disease or a central nervous system (CNS) disease. In some embodiments, the subject is a human patient suffering from an otologic disease selected from the group consisting of prebycusis, prebystatsis, noise-induced hearing loss, Meniere's disease, labyrinthitis, vestibular neuronitis, cochlear otosclerosis, trauma, ototoxic injury, and autoimmune inner ear disease. In some embodiments, the subject is a human patient suffering from a CNS disease selected from the group consisting of congenital disorder, traumatic brain injury, inflammatory disease, infectious disease, neoplastic disease, neurodegenerative disease, vascular disease, seizure disorders, and neuropsychiatric disease.

In some embodiments, the therapeutic and/or diagnostic agent does not comprise N-acetylcysteine (NAC), Acetyl-L-Carnitine, glutathione monoethylester, ebselen, D-methionine, carbamathione, and Szeto-Schiller peptides and their functional analogs. In some embodiments, the therapeutic and/or diagnostic agent does not comprise an antioxidant.

In some embodiments, the 2,4-DSPBN is co-administered or administered sequentially with a diagnostic agent. In some embodiments, the diagnostic agent is selected from the group consisting of gadolinium compounds, contrast agents, radiopharmaceuticals, antisense radiopharmaceuticals, and peptide radiopharmaceuticals.

In some embodiments, the 2,4-DSPBN is co-administered or administered sequentially with a therapeutic agent. In some embodiments, the therapeutic agent is selected from small molecule drugs, peptides, proteins, antibodies, RNAs, DNAs, anti-neoplastics, anti-infectives, anti-inflammatories, steroids, NSAIDs, seizure medications, psychotrophic medications, medications for neurodegenerative diseases, antivirals, metabolic agents, diuretics, antioxidants, reparative agents, and regenerative agents. In some embodiments, the therapeutic agent when co-administered or administered sequentially with 2,4-DSPBN is effective for treating an otologic disease. In some embodiments, the therapeutic agent when co-administered or administered sequentially with 2,4-DSPBN is effective for treating a CNS disease.

Another aspect of the invention relates to a method of treating an otologic disease, comprising administering to a subject in need thereof an effective amount of a therapeutic agent, along with an effective amount of 2,4-DSPBN, said therapeutic agent being characterized as being unable or poorly able, in the absence of said effective amount of 2,4-DSPBN, to cross the blood-cochlear barrier of said subject in an amount sufficient to deliver a therapeutic benefit against the otologic disease.

Another aspect of the invention relates to a method of treating a CNS disease, comprising administering to a subject in need thereof an effective amount of a therapeutic agent, along with an effective amount of 2,4-DSPBN, said therapeutic agent being characterized as being unable or poorly able, in the absence of said effective amount of 2,4-DSPBN, to cross the blood-brain barrier or the blood-cerebrospinal fluid barrier of said subject in an amount sufficient to deliver a therapeutic benefit against the CNS disease.

A further aspect of the invention relates to a method of treating an otologic disease, comprising administering to a subject in need thereof an effective amount of a therapeutic agent, along with an effective amount of 2,4-DSPBN, wherein said therapeutic agent is not an antioxidant, and wherein the presence of said effective amount of 2,4-DSPBN reduces the amount of the therapeutic agent required to deliver a therapeutic benefit against the otologic disease by at least 10%, or at least 20%, or at least 30%, or at least 40%, or at least 50%, or at least 60%, or at least 70%, or at least 80%, or at least 90%.

A further aspect of the invention relates to a method of treating a CNS disease, comprising administering to a subject in need thereof an effective amount of a therapeutic agent, along with an effective amount of 2,4-DSPBN, wherein said therapeutic agent is not an antioxidant, and wherein the presence of said effective amount of 2,4-DSPBN reduces the amount of the therapeutic agent required to deliver a therapeutic benefit against the CNS disease to said subject by at least 10%, or at least 20%, or at least 30%, or at least 40%, or at least 50%, or at least 60%, or at least 70%, or at least 80%, or at least 90%.

A further aspect of the invention relates to a method of transporting a therapeutic and/or diagnostic agent across a blood-brain barrier or a blood-cochlear barrier or a blood-cerebrospinal fluid barrier of a subject in need thereof, comprising administering to a subject in need thereof an effective amount of a therapeutic and/or diagnostic agent, along with an effective amount of 2,4-DSPBN, wherein said therapeutic and/or diagnostic agent is not an antioxidant, and wherein the presence of said effective amount of 2,4-DSPBN reduces the amount of said therapeutic and/or diagnostic agent required to deliver a therapeutic and/or diagnostic benefit to said subject by at least 10%, or at least 20%, or at least 30%, or at least 40%, or at least 50%, or at least 60%, or at least 70%, or at least 80%, or at least 90%.

These and other features, together with the organization and manner of operation thereof, will become apparent from the following detailed description when taken in conjunction with the accompanying drawings.

### BRIEF DESCRIPTION OF THE DRAWINGS

FIG. 1: HPN-07 increases permeability of the blood-brain-barrier (BBB) in rat brains. (Ai) Representative pre-contrast T1-weighted MR image of a rat brain treated with HPN-07 prior to the administration of the MRI contrast agent, Gd-DTPA. (Aii) Representative post-contrast image of a rat brain treated with HPN-07, 10 min following administration of Gd-DTPA. (Aiii) Difference image of image Aii minus image Ai. Note contrast enhanced areas throughout the brain due to increased BBB permeability which allowed Gd-DTPA contrast agent to enter brain tissue. (Bi) Representative pre-contrast T1-weighted MR image of a rat brain not treated with HPN-07 prior to the administration of the MRI contrast agent, Gd-DTPA. (Bii) Representative post-contrast image of a rat brain not treated with HPN-07, 10 min following administration of Gd-DTPA. (Biii) Difference image of image Bii minus image Bi. Note lack of contrast enhanced areas throughout the brain due to no increased BBB permeability which did not allow Gd-DTPA contrast agent to enter brain tissue. (C) Quantitative assessment of the difference in MRI signal intensity (SI) between post-contrast and pre-contrast images for rat brain regions either not treated with HPN-07 (No HPN-07) or treated with HPN-07. White arrow heads in panels “ii” in both “A” and “B” depict blood vessel cross-sections which contribute to MRI signal intensities from the administration of the Gd-DTPA contrast agent.

FIG. 2: Preliminary kinetic MRI data indicate that the percent (%) of MRI signal intensity (SI) increases maximally at ˜2 hours after HPN-07 (OKN-007) injection.

### DETAILED DESCRIPTION

The invention described herein provides a method for facilitating the transport of a therapeutic or diagnostic agent across a blood-brain barrier (BBB) or a blood-cochlear barrier (BCB) or a blood-cerebrospinal fluid barrier (BCSFB) of a subject, by co-administering an effective amount of 2,4-disulfonyl α-phenyl tertiary butyl nitrone (2,4-DSPBN). The presence of the effective amount of 2,4-DSPBN either enables the therapeutic or diagnostic agent to cross the BBB or the BCB or the BCSFB in an amount sufficient to deliver a therapeutic or diagnostic benefit to the subject, or reduces the amount of the therapeutic or diagnostic agent required to deliver a therapeutic or diagnostic benefit to the subject.

### 2,4-disulfonyl α-phenyl tertiary butyl nitrone (2,4-DSPBN)

2,4-disulfonyl α-phenyl tertiary butyl nitrone is also referred to as 2,4-disulfonyl PBN, 2,4-DSPBN, NXY-059 or HPN-07. It has the following structure:

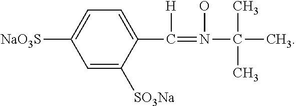

The acid form of the compound has the following structure:

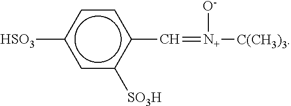

The acid form may be a solid or found in low pH solutions. The ionized salt form of the compound exists at higher pH and may be represented by either of the following structures:

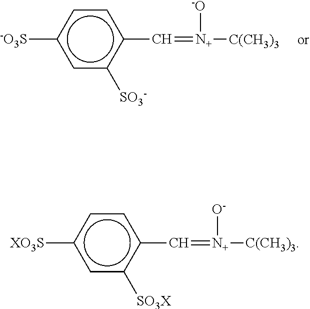

In the salt form, X is a pharmaceutically acceptable cation. Most commonly, this cation is a monovalent material such as sodium, potassium or ammonium, but it can also be a multivalent alone or cation in combination with a pharmaceutically acceptable monovalent anion, for example calcium with a chloride, bromide, iodide, hydroxyl, nitrate, sulfonate, acetate, tartrate, oxalate, succinate, pamoate or the like anion; magnesium with such anions; zinc with such anions or the like. Among these materials, the free acid and the simple sodium, potassium or ammonium salts are most preferred with the calcium and magnesium salts also being preferred but somewhat less so. 2,4-DSPBN is described in detail by U.S. Pat. No. 5,488,145, which is incorporated herein by reference. The salts of 2,4-DSPBN may also be used for facilitating the transport of therapeutic or diagnostic agents across the BBB or the BCB or the BCSFB in a manner similar to the use of 2,4-DSPBN as described herein.

To open up the BBB and facilitate the transport of at least one therapeutic or diagnostic agent across the BBB, 2,4-DSPBN can be administered at a dose of, for example, between about 1 mg/kg to about 500 mg/kg body weight, or between about 5 mg/kg to about 400 mg/kg body weight, or between about 10 mg/kg to about 300 mg/kg body weight, or at about 10 mg/kg body weight, or at about 20 mg/kg body weight, or at about 50 mg/kg body weight, or at about 100 mg/kg body weight, or at about 150 mg/kg body weight, or at about 200 mg/kg body weight, or at about 250 mg/kg body weight, or at about 300 mg/kg body weight. The subject can be administered one dose daily, or two doses daily, or three doses daily, or four doses daily, or five doses daily.

To open up the BCB and facilitate the transport of at least one therapeutic or diagnostic agent across the BCB, 2,4-DSPBN can be administered at a dose of, for example, between about 1 mg/kg to about 500 mg/kg body weight, or between about 5 mg/kg to about 400 mg/kg body weight, or between about 10 mg/kg to about 300 mg/kg body weight, or at about 10 mg/kg body weight, or at about 20 mg/kg body weight, or at about 50 mg/kg body weight, or at about 100 mg/kg body weight, or at about 150 mg/kg body weight, or at about 200 mg/kg body weight, or at about 250 mg/kg body weight, or at about 300 mg/kg body weight. The subject can be administered one dose daily, or two doses daily, or three doses daily, or four doses daily, or five doses daily.

To open up the BCSFB and facilitate the transport of at least one therapeutic or diagnostic agent across the BCSFB, 2,4-DSPBN can be administered at a dose of, for example, between about 1 mg/kg to about 500 mg/kg body weight, or between about 5 mg/kg to about 400 mg/kg body weight, or between about 10 mg/kg to about 300 mg/kg body weight, or at about 10 mg/kg body weight, or at about 20 mg/kg body weight, or at about 50 mg/kg body weight, or at about 100 mg/kg body weight, or at about 150 mg/kg body weight, or at about 200 mg/kg body weight, or at about 250 mg/kg body weight, or at about 300 mg/kg body weight. The subject can be administered one dose daily, or two doses daily, or three doses daily, or four doses daily, or five doses daily.

Other Nitrone Compounds that Open Up the BBB/BCB/BCSFB

In place of or in addition to 2,4-DSPBN, other nitrone compounds can also be used to open up the BBB/BCB/BCSFB of a subject. In some embodiments, the nitrone compound is selected from phenyl butyl nitrone (PBN) and its derivatives. In some embodiments, the nitrone compound is PBN. In some embodiments, the nitrone compound is 4-hydroxy-α-phenyl butyl nitrone (4-OHPBN). In some embodiments, the nitrone compound is 2-sulfonyl-α-phenyl tertiary butyl nitrone (S-PBN).

Therefore, the present application expressly covers the use of any of the aforementioned nitrone compounds in place of or in addition to 2,4-DSPBN in all embodiments disclosed herein. Hence, methods are disclosed in which one or more of phenyl butyl nitrone (PBN), 4-hydroxy-α-phenyl butyl nitrone (4-OHPBN) and 2-sulfonyl-α-phenyl tertiary butyl nitrone (S-PBN) are used in place of or in addition to the 2,4-DSPBN.

Administration of 2,4-DSPBN with a Therapeutic and Diagnostics Agent

2,4-DSPBN can be administrated with at least one therapeutic or diagnostic agent to facilitate the transport of therapeutic or diagnostic agent across the BBB or the BCB or the BCSFB. In some embodiments, 2,4-DSPBN and the diagnostic or therapeutic agent are co-administered as a mixture. In some embodiments, the 2,4-DSPBN and the diagnostic or therapeutic agent are co-administered as a covalently- or noncovalently-bound conjugate. In some embodiments, 2,4-DSPBN and the diagnostic or therapeutic agent are administered sequentially as distinct dosage forms.

In some embodiments, the method comprises co-administering or administering sequentially, in any order, 2,4-DSPBN and a diagnostic agent. In some embodiments, the method comprises co-administering or administering sequentially, in any order, 2,4-DSPBN and a therapeutic agent, (c) 2,4-DSPBN and a diagnostic agent. In some embodiments, the method comprises co-administering or administering sequentially, in any order, 2,4-DSPBN and a diagnostic agent and a therapeutic agent. In some embodiments, the method comprises co-administering or administering sequentially, in any order, 2,4-DSPBN and a theranostic (diagnostic and therapeutic) agent.

In some embodiments, 2,4-DSPBN and the diagnostic or therapeutic agent are administered orally. Other delivery methods including, but not limited to, intravenously, subcutaneously, by inhalation, sublingually, subdermally, intrathecally, or locally within the ear. Further the active composition may be administered as a nanoparticle or dendrimer formulation. The nanoparticle may be multifunctional and composed of a polymer and paramagnetic iron oxide particles to allow the application of external magnetic forces to aid in the delivery of the drug to the desired target such as the inner ear or the dorsal cochlear nucleus. Additionally, the composition may be formulated with additives known to those skilled in the art to enhance oral absorption and alter bioavailability kinetics.

In some embodiments, the therapeutic or diagnostic agent can be characterized as being unable or poorly able, in the absence of an effective amount of 2,4-DSPBN, to cross the BBB of the subject in an amount sufficient to deliver a therapeutic or diagnostic benefit to the subject. In some embodiments, less than 5%, or less than 2%, or less than 1%, or less than 0.5%, or less than 0.2%, or less than 0.1% of the therapeutic or diagnostic agent in the blood is able to cross the BBB of the subject in the absence of 2,4-DSPBN.

In some embodiments, the co-administration of 2,4-DSPBN with the therapeutic or diagnostic agent can increase the permeability of the therapeutic or diagnostic agent across the BBB by at least 10%, or at least 20%, or at least 50%, or at least 100%, or at least 200%, or at least 500%. In other words, when a given amount of the therapeutic or diagnostic agent is co-administered with 2,4-DSPBN, at least 10%, or at least 20%, or at least 50%, or at least 100%, or at least 200%, or at least 500% more of the therapeutic or diagnostic agent will be able to cross the BBB from the blood circulation of the subject, compared to when said given amount of the therapeutic or diagnostic agent is administered in the absence of 2,4-DSPBN.

In some embodiments, the therapeutic or diagnostic agent can be characterized as being unable or poorly able, in the absence of an effective amount of 2,4-DSPBN, to cross the BCB of the subject in an amount sufficient to deliver a therapeutic or diagnostic benefit to the subject. In some embodiments, less than 5%, or less than 2%, or less than 1%, or less than 0.5%, or less than 0.2%, or less than 0.1% of the therapeutic or diagnostic agent in the blood is able to cross the BCB of the subject in the absence of 2,4-DSPBN.

In some embodiments, the co-administration of 2,4-DSPBN with the therapeutic or diagnostic agent can increase the permeability of the therapeutic or diagnostic agent across the BCB by at least 10%, or at least 20%, or at least 50%, or at least 100%, or at least 200%, or at least 500%. In other words, when a given amount of the therapeutic or diagnostic agent is co-administered with 2,4-DSPBN, at least 10%, or at least 20%, or at least 50%, or at least 100%, or at least 200%, or at least 500% more of the therapeutic or diagnostic agent will be able to cross the BCB from the blood circulation of the subject, compared to when said given amount of the therapeutic or diagnostic agent is administered in the absence of 2,4-DSPBN.

In some embodiments, the therapeutic or diagnostic agent can be characterized as being unable or poorly able, in the absence of an effective amount of 2,4-DSPBN, to cross the BCSFB of the subject in an amount sufficient to deliver a therapeutic or diagnostic benefit to the subject. In some embodiments, less than 5%, or less than 2%, or less than 1%, or less than 0.5%, or less than 0.2%, or less than 0.1% of the therapeutic or diagnostic agent in the blood is able to cross the BCSFB of the subject in the absence of 2,4-DSPBN.

In some embodiments, the co-administration of 2,4-DSPBN with the therapeutic or diagnostic agent can increase the permeability of the therapeutic or diagnostic agent across the BCSFB by at least 10%, or at least 20%, or at least 50%, or at least 100%, or at least 200%, or at least 500%. In other words, when a given amount of the therapeutic or diagnostic agent is co-administered with 2,4-DSPBN, at least 10%, or at least 20%, or at least 50%, or at least 100%, or at least 200%, or at least 500% more of the therapeutic or diagnostic agent will be able to cross the BCSFB from the blood circulation of the subject, compared to when said given amount of the therapeutic or diagnostic agent is administered in the absence of 2,4-DSPBN.

In some embodiments, 2,4-DSPBN is co-administered with a diagnostic agent. The diagnostic agent can be, for example, gadolinium compounds, contrast agents, radiopharmaceuticals, antisense radiopharmaceuticals, and peptide radiopharmaceuticals.

In some embodiments, 2,4-DSPBN is co-administered with a therapeutic agent. In some embodiments, the therapeutic agent when co-administered with 2,4-DSPBN is effective for treating a CNS disease. The therapeutic agent can be, for example, small molecule drugs, peptides, proteins, antibodies, RNAs (e.g., antisense therapy, RNAi therapy), DNAs (e.g., gene therapy, CRISPR therapy), anti-neoplastics, anti-infectives, anti-inflammatories (e.g., steroids, NSAIDs), seizure medications, psychotrophic medications, and medications for neurodegenerative diseases. In some embodiments, the therapeutic agent when co-administered with 2,4-DSPBN is effective for treating an otologic disease. The therapeutic agent can be, for example, anti-infectives (e.g., antivirals like famciclovir), anti-inflammatories (e.g., steroids like dexamethasone, methyl prednisolone, etc.), metabolic agents (e.g., diuretics), antioxidants, reparative agents, and regenerative agents (e.g., small molecules, siRNA, DNA, peptides).

Examples of therapeutic agents suitable for co-administration with 2,4-DSPBN include riluzole (e.g., for the treatment of amyotrophic lateral sclerosis), interferon beta 1-A (e.g., for the treatment of multiple sclerosis), carbamazepine (e.g., for the treatment of epilepsy), c (e.g., for the treatment of epilepsy and/or migraine), donepezil (e.g., for the treatment of Alzheimer's Disease), zolmitriptan (e.g., for the treatment of migraine), topiramate (e.g., for the treatment of epilepsy and/or seizure), ropinirole (e.g., for the treatment of Parkinson's Disease), pramipexole (e.g., for the treatment of Parkinson's Disease), dihydroergotamine (e.g., for the treatment of migraine), sumatriptan (e.g., for the treatment of migraine), glatiramer acetate (e.g., for the treatment of multiple sclerosis), carbamazepine (e.g., for the treatment of epilepsy), tolcapone (e.g., for the treatment of Parkinson's Disease), rizatriptan (e.g., for the treatment of migraine), lamotrigine (e.g., for the treatment of epilepsy), pemoline (e.g., for the treatment of ADHD), naratriptan (e.g., for the treatment of migraine), zaleplon (e.g., for the treatment of insomnia), levetiracetam (e.g., for the treatment of epilepsy), entacapone (e.g., for the treatment of Parkinson's Disease), zonisamide (e.g., for the treatment of epilepsy and Parkinson's Disease), oxcarbazepine (e.g., for the treatment of epilepsy), mitoxantrone (e.g., for the treatment of multiple sclerosis), gabapentin (e.g., for the treatment of epilepsy), rivastigmine (e.g., for the treatment of dementia related to Alzheimer's disease or Parkinson's Disease), galantamine (e.g., for the treatment of Alzheimer's disease), methylphenidate (e.g., for the treatment of ADHD), frovatriptan (e.g., for the treatment of migraine), dexmethylphenidate (e.g., for the treatment of ADHD), almotriptan (e.g., for the treatment of migraine), atomoxetine (e.g., for the treatment of ADHD), eletriptan (e.g., for the treatment of migraine), memantine (e.g., for the treatment of Alzheimer's disease), eszopiclone (e.g., for the treatment of insomnia), apomorphine (e.g., for the treatment of Parkinson's Disease), natalizumab (e.g., for the treatment of multiple sclerosis), ramelteon (e.g., for the treatment of insomnia), paliperidone (e.g., for the treatment of schizophrenia), lisdexamfetamine (e.g., for the treatment of ADHD), armodafinil (e.g., for the treatment of sleep disorder), rotigotine (e.g., for the treatment of Parkinson's Disease), tetrabenazine (e.g., for the treatment of chorea due to Huntington's disease), lacosamide (e.g., for the treatment of partial-onset seizure), valproate (e.g., for the treatment of epilepsy and bipolar disorder), rufinamide (e.g., for the treatment of seizure associated with Lennox-Gastaut syndrome), vigabatrin (e.g., for the treatment of epilepsy), guanfacine (e.g., for the treatment of ADHD), zolpidem (e.g., for the treatment of insomnia), diclofenac (e.g., for the treatment of migraine), caprylidene (e.g., for the treatment of Alzheimer's disease), doxepin (e.g., for the treatment of insomnia), clonidine (e.g., for the treatment of ADHD), onabotulinumtoxinA (e.g., for the treatment of migraine), dalfampridine (e.g., for the treatment of multiple sclerosis), vilazodone (e.g., for the treatment of major depressive disorder), ezogabine (e.g., for the treatment of partial-onset seizure), clobazam (e.g., for the treatment of seizure associated with Lennox-Gastaut syndrome), perampanel (e.g., for the treatment of partial-onset seizure), nimodipine (e.g., for the prevention of cerebral vasospasm and resultant ischemia), levomilnacipran (e.g., for the treatment of major depressive disorder), droxidopa (e.g., for the treatment of neurogenic orthostatic hypotension associated with multiple system atrophy, familial amyloid polyneuropathy, pure autonomic failure, and Parkinson's Disease), licarbazepine (e.g., for the treatment of partial-onset seizure), alemtuzumab (e.g., for the treatment of multiple sclerosis), tasimelteon (e.g., for the treatment of non-24-hour sleep-wake disorder), suvorexant (e.g., for the treatment of insomnia), carbidopa/levodopa (e.g., for the treatment of Parkinson's Disease), everolimus (e.g., for the treatment of brain cancer), bevacizumab (e.g., for the treatment of brain cancer), carmustine (e.g., for the treatment of brain cancer), lomustine (e.g., for the treatment of brain cancer), temozolomide (e.g., for the treatment of brain cancer), and pharmaceutical acceptable salts thereof.

It is believed that N-acetylcysteine (NAC) crosses the BBB of its own accord and, hence, the therapeutic or diagnostic agent described herein should be construed to exclude NAC. To the extent that an express exclusion is required for the sake of clarity, then the following phrase is provided herein, “provided that a combination therapy comprising NAC and 2,4-DSPBN is specifically excluded.”

In some embodiments, the therapeutic or diagnostic agent specifically excludes any antioxidant. In some embodiments, the therapeutic or diagnostic agent specifically excludes any one of N-acetylcysteine, Acetyl-L-Carnitine, glutathione monoethylester, ebselen, D-methionine, carbamathione, and Szeto-Schiller peptides and their functional analogs.

Otologic Diseases and Central Nervous System Diseases

In some embodiments, 2,4-DSPBN and the diagnostic or therapeutic agent are co-administered to a human patient suffering from a central nervous system (CNS) disease. Examples of the CNS disease include congenital disorder, traumatic brain injury (e.g., closed head trauma, penetrating head trauma, blast-induced head trauma, concussion), inflammatory disease, infectious disease (e.g., meningitis, cerebritis/encephalitis, brain abscess), neoplastic disease (e.g., CNS malignancies), neurodegenerative disease (e.g., Alzheimer's Disease, Parkinson's Disease, multiple sclerosis (MS), amyotrophic lateral sclerosis (ALS), stroke associated neurodegeneration), vascular disease (e.g., ischemic stroke, hemorrhagic stroke), seizure disorders (e.g., epilepsy), and neuropsychiatric disease (e.g., depression, bipolar disorder, schizophrenia), migraine, and attention deficit/hyperactivity disorder (ADHD).

In some embodiments, 2,4-DSPBN and the diagnostic or therapeutic agent are co-administered to a human patient suffering from an otologic disease. Examples of the otologic disease include prebycusis, prebystatsis, noise-induced hearing loss (e.g., acute noise-induced hearing loss, chronic noise-induced hearing loss), Meniere's disease, labyrinthitis (e.g., viral-induced labyrinthitis, bacterial-induced labyrinthitis), vestibular neuronitis, cochlear otosclerosis, trauma, ototoxic injury (e.g., cochlear injury, labyrinth injury), and autoimmune inner ear disease.

In one embodiment, 2,4-DSPBN and the diagnostic or therapeutic agent are co-administered to a human patient suffering from acute acoustic trauma (AAT), provided that a combination therapy comprising NAC and 2,4-DSPBN is specifically excluded. AAT is known to cause permanent hearing loss. Hearing loss from AAT is also enhanced by simultaneous exposure to other toxins such as low levels of carbon monoxide or acrylonitrile. Recent studies indicate that free radical processes are involved in the AAT-induced hearing loss.

In one embodiment, 2,4-DSPBN and the diagnostic or therapeutic agent are co-administered to a human patient suffering from traumatic brain injury (TBI), provided that a combination therapy comprising NAC and 2,4-DSPBN is specifically excluded. There is increased evidence that blast overpressure is transmitted across the skull into the brain. This sets up the potential to cause TBI including damage to the central auditory centers of the brain, e.g. brainstem, temporal lobe, and thalamus which could explain symptoms such as hearing loss, dizziness, and tinnitus. Of particular significance is the observation that blast-related TBI produces significantly greater rates of hearing loss and tinnitus (60%) compared with non-blast related TBI. Similarly, intense sound- or noise-induced changes in the central auditory structure have been reported, including the cochlear nucleus, inferior colliculus, medial geniculate body and primary auditory cortex. Although some mechanical damage will have permanent effects, much of the long-term damage results from secondary molecular and cellular processes that are triggered by the blast-induced trauma amplify the effects of mechanical damage. TBI initiates an almost immediate injury process including contusion, diffuse axonal injury, hematoma, subarachnoid hemorrhage followed shortly thereafter by a variety of secondary injuries. The secondary injuries can include ischemia, edema, oxidative damage, decreased ATP, cytoskeleton changes, inflammation, and activation of cell death pathways.

A further embodiment of the invention relates to a method for treating AAT, comprising co-administering to a subject in need thereof an effective amount of a therapeutic agent against AAT, along with an effective amount of 2,4-DSPBN, wherein the therapeutic agent is not an antioxidant, and wherein the presence of the effective amount of 2,4-DSPBN either reduces the amount of the therapeutic agent required to deliver a therapeutic benefit to the subject against AAT or enables the therapeutic agent to cross the BCB in an amount sufficient to deliver a therapeutic benefit to the subject against AAT, provided that a combination therapy comprising NAC and 2,4-DSPBN is specifically excluded.

A further embodiment of the invention relates to a method for treating TBI, comprising co-administering to a subject in need thereof an effective amount of a therapeutic agent against TBI, along with an effective amount of 2,4-DSPBN, wherein the therapeutic agent is not an antioxidant, and wherein the presence of the effective amount of 2,4-DSPBN either reduces the amount of the therapeutic agent required to deliver a therapeutic benefit to the subject against TBI or enables the therapeutic agent to cross the BBB or the BCSFB in an amount sufficient to deliver a therapeutic benefit to the subject against TBI, provided that a combination therapy comprising NAC and 2,4-DSPBN is specifically excluded.

A further embodiment of the invention relates to a method for treating Alzheimer's Disease, comprising co-administering to a subject in need thereof an effective amount of a therapeutic agent against Alzheimer's Disease, along with an effective amount of 2,4-DSPBN, wherein the therapeutic agent is not an antioxidant, and wherein the presence of the effective amount of 2,4-DSPBN either reduces the amount of the therapeutic agent required to deliver a therapeutic benefit to the subject against Alzheimer's Disease or enables the therapeutic agent to cross the BBB or the BCSFB in an amount sufficient to deliver a therapeutic benefit to the subject against Alzheimer's Disease.

A further embodiment of the invention relates to a method for treating Parkinson's Disease, comprising co-administering to a subject in need thereof an effective amount of a therapeutic agent against Parkinson's Disease, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against Parkinson's Disease or enables the therapeutic agent to cross the BBB or the BCSFB in an amount sufficient to deliver a therapeutic benefit to the subject against Parkinson's Disease.

A further embodiment of the invention relates to a method for treating MS, comprising co-administering to a subject in need thereof an effective amount of a therapeutic agent against MS, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against MS or enables the therapeutic agent to cross the BBB or the BCSFB in an amount sufficient to deliver a therapeutic benefit to the subject against MS.

A further embodiment of the invention relates to a method for treating ALS, comprising co-administering to a subject in need thereof an effective amount of a therapeutic agent against ALS, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against ALS or enables the therapeutic agent to cross the BBB or the BCSFB in an amount sufficient to deliver a therapeutic benefit to the subject against ALS.

A further embodiment of the invention relates to a method for treating epilepsy or seizure, comprising co-administering to a subject in need thereof an effective amount of a therapeutic agent against epilepsy or seizure, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against epilepsy or seizure or enables the therapeutic agent to cross the BBB or the BCSFB in an amount sufficient to deliver a therapeutic benefit to the subject against epilepsy or seizure.

A further embodiment of the invention relates to a method for treating depression, bipolar disorder or schizophrenia, comprising co-administering to a subject in need thereof an effective amount of a therapeutic agent against depression, bipolar disorder or schizophrenia, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against depression, bipolar disorder or schizophrenia or enables the therapeutic agent to cross the BBB or the BCSFB in an amount sufficient to deliver a therapeutic benefit to the subject against depression, bipolar disorder or schizophrenia.

A further embodiment of the invention relates to a method for treating ADHD, comprising co-administering to a subject in need thereof an effective amount of a therapeutic agent against ADHD, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against ADHD or enables the therapeutic agent to cross the BBB or the BCSFB in an amount sufficient to deliver a therapeutic benefit to the subject against ADHD.

A further embodiment of the invention relates to a method for treating migraine, comprising co-administering to a subject in need thereof an effective amount of a therapeutic agent against migraine, along with an effective amount of 2,4-DSPBN, wherein said therapeutic agent is not an antioxidant, and wherein the presence of the effective amount of 2,4-DSPBN either reduces the amount of the therapeutic agent required to deliver a therapeutic benefit to the subject against migraine or enables the therapeutic agent to cross the BBB or the BCSFB in an amount sufficient to deliver a therapeutic benefit to the subject against migraine.

A further embodiment of the invention relates to a method for treating a brain cancer, comprising co-administering to a subject in need thereof an effective amount of a therapeutic agent against the brain cancer, along with an effective amount of 2,4-DSPBN, and wherein the presence of the effective amount of 2,4-DSPBN either reduces the amount of the therapeutic agent required to deliver a therapeutic benefit to the subject against the brain cancer or enables the therapeutic agent to cross the BBB or the BCSFB in an amount sufficient to deliver a therapeutic benefit to the subject against the brain cancer.

A further embodiment of the invention relates to a method for treating a neuronopathic lysosomal storage disease, comprising co-administering to a subject in need thereof an effective amount of a therapeutic agent against the neuronopathic lysosomal storage disease, along with an effective amount of 2,4-DSPBN, and wherein the presence of the effective amount of 2,4-DSPBN either reduces the amount of the therapeutic agent required to deliver a therapeutic benefit to the subject against the neuronopathic lysosomal storage disease or enables the therapeutic agent to cross the BBB or the BCSFB in an amount sufficient to deliver a therapeutic benefit to the subject against the neuronopathic lysosomal storage disease.

### WORKING EXAMPLES

### Example 1

Contrast-enhanced magnetic resonance imaging (CE-MRI). A group of rats (n=9) were treated with an average of 300 mg/kg HPN-07 (APAC Pharmaceuticals LLC, Columbia, Md.) for three days at a concentration of 4.7 mg/mL in their drinking water prior to MRI assessment. CE-MRI experiments were carried out under general anaesthesia (1-2% Isoflurane, 0.8-1.0 L/min O₂) on a Bruker Biospec 7T/30 cm horizontal imaging spectrometer. Rats were restrained by anaesthesia (2% isoflurane), were placed in an MR probe, and their brains were localised by MRI. Images were obtained using a Bruker S116 gradient coil (2.0 mT/m/A) and a 72 mm quadrature multi-rung RF coil. Multiple ¹H-MR image slices were taken in the axial plane using a T₁-weighted spin echo multislice sequence; repetition time 2642.7 ms, echo time 17.46 ms, 192×192 matrix, 2 steps per acquisition, 3.5×3.5 cm² field of view, 1 mm slice thickness). Rat brains were imaged at 0 (pre-contrast) and at 10-30 min post-contrast agent injection. Rats were injected i.v. with a MRI contrast agent, Gd-DTPA (gadolinium diethylene triamine penta acetic acid, 0.4 mmol Gd⁺³/kg). Relative MR signal intensities were calculated for selected regions of interest in rat brain areas. For each animal, the signal intensity measurement before contrast and then 10-30 min after contrast were used to obtain the percent increase in signal intensity. As a group, sham control animals had a signal intensity value of 0.48%, which was designated as the baseline permeability for the contrast agent.

Statistical analysis. All parameters measured are expressed as means±standard error of the mean (SEM). One-way ANOVA (SPSS 14.0 for windows) was used to determine if there were statistically significant differences among the three experimental groups (NC, B, and B/T) at each time point. When a significant difference among groups was found, a post hoc test (Tukey HSD) was used to determine if there were statistically significant differences between group pairings (i.e. NC vs. B; NC vs. B/T; B vs. B/T at each time point). Statistical analyses were conducted using GraphPad Prism 4 software (GraphPad Software, Inc., La Jolla, Calif.). Student's t-test (two-tailed) was used to determine if there was statistically significant difference in MRI signal intensity between Gd-DTPA pre- and post-contrast. A p-value of less than 0.05 was considered to be significant.

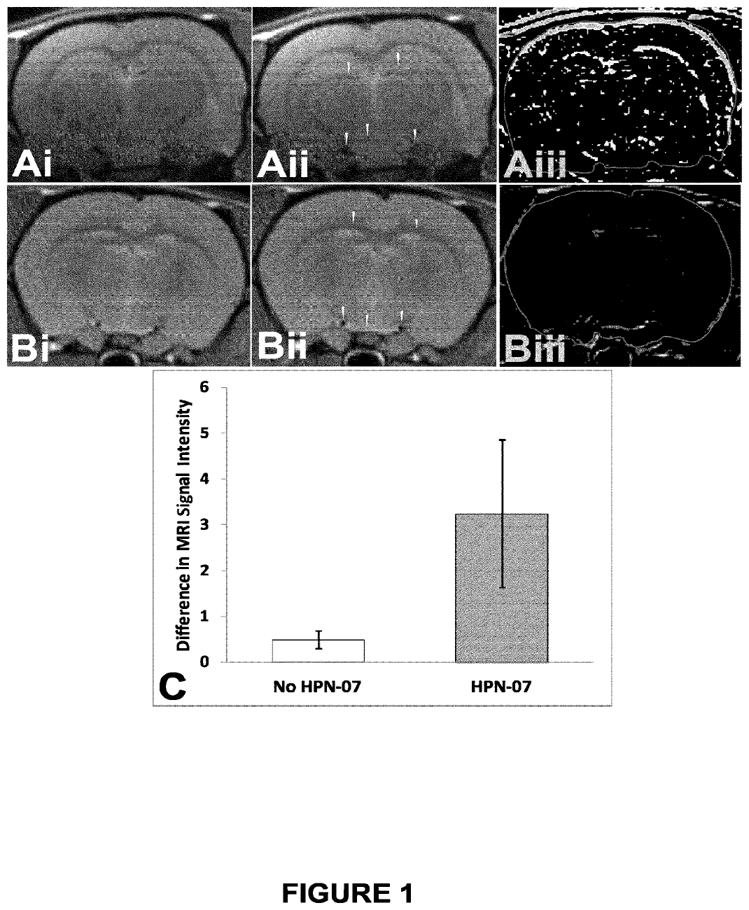

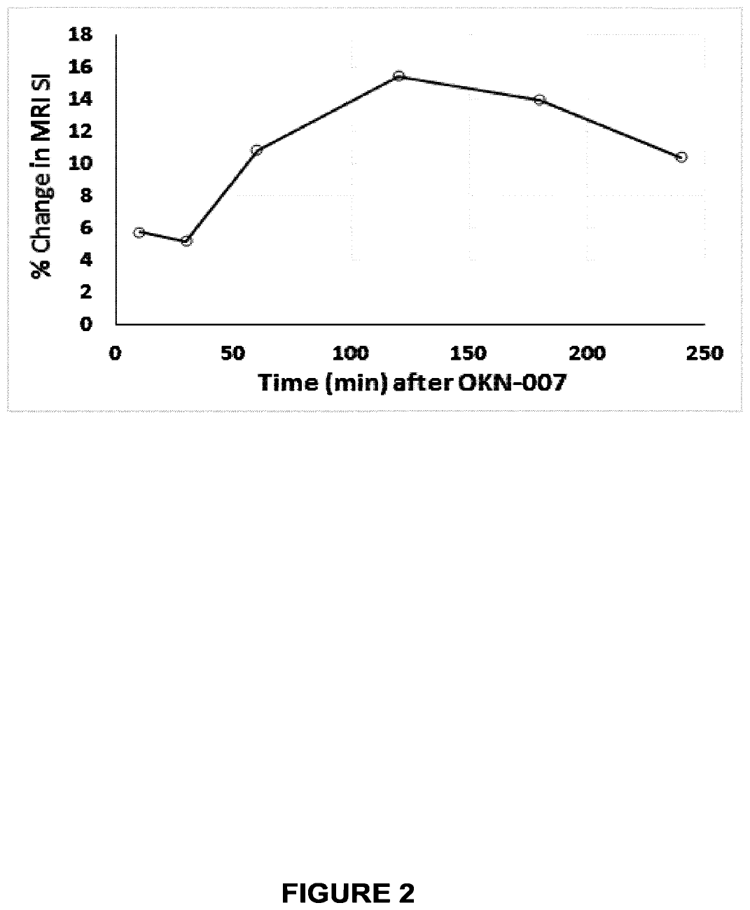

Assessment of whether HPN-07 affects the blood-brain barrier (BBB). The influence of HPN-07 on potentially altering access to the brain extracellular fluid was previously unknown. Accordingly, CE-MRI was used to establish whether HPN-07 may temporarily open up the BBB to facilitate drug delivery to the brain. Gd-DTPA is a commonly used MRI contrast agent that does not cross the BBB in a normal brain. From this analysis, it was found that there was a significant (approximately seven-fold) increase (p<0.01) in post-contrast signal intensity following administration of the MRI contrast agent gadolinium diethylene triamine pentacetate (Gd-DTPA) in rats treated with HPN-07 compared to sham controls, indicating enhanced penetrance across the BBB (FIG. 1). Moreover, preliminary kinetic MRI data indicate that the percent (%) of MRI signal intensity (SI) increases maximally at ˜2 hours after HPN-07 (OKN-007) injection (FIG. 2).

This shows that HPN-07 can temporarily open up the BBB, which allows other therapeutic and diagnostic agents that normally cannot effectively cross the BBB to enter the brain. The ability of HPN-07 to facilitate penetrance of antioxidants or other drug formulations across the BBB, and the BCB by analogy, will have considerable therapeutic benefit for enhancing the efficacy of treatment strategies aimed at targeting neurodegenerative disorders or tumors of the brain as well as inner ear injuries.

As used herein, the singular terms “a,” “an,” and “the” include plural referents unless the context clearly dictates otherwise. Thus, for example, reference to a compound can include multiple compounds unless the context clearly dictates otherwise.

As used herein, the terms “substantially,” “substantial,” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations, which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scopes of this invention.

What is claimed is:

1. A method for treating an otologic disease comprising:
delivering to an organism experiencing an otologic disease an amount of a composition comprising 2,4-DSPBN sufficient to increase the permeability of the blood-cochlear barrier;
delivering to the organism a therapeutic amount of an agent having effectiveness in treating the otologic disease.

2. The method of claim 1, wherein the otologic disease is selected from the group consisting of: prebycusis, prebystatsis, noise-induced hearing loss, Meniere's disease, labyrinthitis, vestibular neuronitis, cochlear otosclerosis, trauma, ototoxic injury, and autoimmune inner ear disease.

3. The method of claim 1, wherein said step of delivering to the organism a therapeutic amount of an agent having effectiveness in treating the otologic disease occurs simultaneously with the step of delivering said composition comprising 2,4-DSPBN.

4. The method of claim 1, wherein said step of delivering to the organism a therapeutic amount of an agent having effectiveness in treating the otologic disease occurs sequentially after the step of delivering said composition comprising 2,4-DSPBN.

5. The method of claim 1, wherein the step of delivering the composition comprising 2,4-DSPBN increases the permeability of the blood cochlear barrier to said therapeutic agent by at least 10%.

6. The method of claim 1, wherein the step of delivering the composition comprising 2,4-DSPBN delivers from about 1 mg/kg of organism body weight to about 500 mg/kg of organism body weight of 2,4-DSPBN.

7. The method of claim 1, wherein the therapeutic agent is selected from the group consisting of: anti-neoplastics, anti-infectives, anti-inflammatories, metabolic agents, diuretics, antioxidants, siRNA, DNA.

8. A method of increasing the permeability of a blood-cochlear barrier of an organism to a therapeutic agent, said therapeutic agent characterized by less than 5% of said therapeutic agent will normally pass through said blood-cochlear barrier, comprising:
delivering to an organism an amount of a composition comprising 2,4-DSPBN sufficient to increase the permeability of the blood-cochlear barrier to said therapeutic agent;
whereby at least 10% more of said therapeutic agent will pass through the blood-cochlear barrier than will pass in the absence of said composition comprising 2,4-DSPBN.

9. The method of claim 8, wherein said therapeutic agent is selected from the group consisting of: small molecule drugs, peptides, proteins, antibodies, RNAs, DNAs, anti-neoplastics, anti-infectives, anti-inflammatories, steroids, NSAIDs, seizure medications, psychotrophic medications, medications for neurodegenerative diseases, antivirals, metabolic agents, diuretics, antioxidants, reparative agents, and regenerative agents.

10. The method of claim 8, wherein said therapeutic agent is characterized by less than 1% of said therapeutic agent will normally pass through said blood-cochlear barrier and wherein said step of delivering to an organism an amount of a composition comprising 2,4-DSPBN sufficient to increase the permeability of the blood-cochlear barrier to said therapeutic agent provides for at least 20% more of said therapeutic agent will pass through the blood-cochlear barrier.

11. A method of increasing the permeability of a blood-cochlear barrier of an organism to a diagnostic agent, said diagnostic agent characterized by less than 5% of said diagnostic agent will normally pass through said blood-cochlear barrier, comprising:
delivering to an organism an amount of a composition comprising 2,4-DSPBN sufficient to increase the permeability of the blood-cochlear barrier to said diagnostic agent;
whereby at least 10% more of said diagnostic agent will pass through the blood-cochlear barrier than will pass in the absence of said composition comprising 2,4-DSPBN.

12. The method of claim 11, wherein said diagnostic agent is selected from the group consisting of: gadolinium compounds, contrast agents, radiopharmaceuticals, antisense radiopharmaceuticals, and peptide radiopharmaceuticals.

13. The method of claim 11, wherein said diagnostic agent is characterized by less than 1% of said diagnostic agent will normally pass through said blood-cochlear barrier and wherein said step of delivering to an organism an amount of a composition comprising 2,4-DSPBN sufficient to increase the permeability of the blood-cochlear barrier to said diagnostic agent provides for at least 20% more of said diagnostic agent will pass through the blood-cochlear barrier.
